(19) United States
(12) Patent Application Publication
(10) Pub. No.: US 2008/0195207 A1
(43) Pub. Date: Aug. 14, 2008

(54) Medical implant

(75) Inventors: Kwan-Ku Lin, Taipei (TW); Chih I. Lin, Taipei (TW)

Correspondence Address: BACON & THOMAS, PLLC, 625 SLATERS LANE, FOURTH FLOOR, ALEXANDRIA, VA, 22314, US

(73) Assignee: A-Spine Holding Group Corp., Taipei (TW)

(21) Appl. No.: 11/808,026

(22) Filed: Jun. 6, 2007

(30) Foreign Application Priority Data
Feb. 9, 2007 (TW) 96104911

(51) Int. Cl. A61F 2/44; A61B 19/00

(52) U.S. Cl. 623/17.12; 606/192

(57) ABSTRACT

A medical implant is disclosed which includes: a flexible container, a proximal end thereof being an open end, the flexible container having one layer of flexible surrounding wall or plural layers of flexible surrounding walls, each surrounding wall being provided with a plurality of through holes thereon through which an interior of the flexible container is in fluid communication with an exterior of the flexible container; a tubular fitting being connected to the open end of the flexible container for inserting or infusing a medical filling material into the flexible container through the tubular fitting; and a thrust part connected to the flexible surrounding wall(s) of the flexible container and at a proximal end of the flexible container.

### FIELD OF THE INVENTION

The present invention is related to a medical implant, and particularly to a medical implant with a flexible container combined with a thrust part.

### BACKGROUND OF THE INVENTION

U.S. Pat. No. 6,719,773 discloses an expandable structure for deployment in interior body regions, in which a complicated wrapping-deploying mechanism (legend 56 in FIGS. 3, 5, 6, 9, 10-17 of U.S. Pat. No. 6,719,773) is needed to insert the expandable structure into the vertebra body through a channel pre-formed. First, the structure is wrapped into a wrapped condition to facilitate the insertion of the structure into the vertebra body through the channel, avoiding the structure from being stuck in the channel. Next, the wrapping-deploying mechanism is used to restore the structure to its original shape for carrying out subsequent steps. Please refer to FIGS. 9-12 and related description in U.S. Pat. No. 6,719,773 for details.

US patent publication Nos. 2004/0073308 and 2006/0149379 disclose a porous mesh device without a wrapping-deploying mechanism. However, in order to avoid the expandable porous mesh device from being stuck in the channel, obstructing the performance of the subsequent steps, a cavity needs to be created in advance in the vertebral body (referring to FIGS. 2-15 of the two publications), the femoral head (referring to FIG. 16-18 of the two publications), or the tibia plateau (referring to FIG. 19-21 of the two publication), thereby the porous mesh device can be pushed forward after being stuck in the channel. Thus, the porous mesh device is deployed in the cavity and then retracted to a pre-determined position, thereby completing a deploying step of the porous mesh device. Since the application of this porous mesh device needs to create a cavity in a bone under treatment, the operation time is lengthened and the recovery time after operation is prolonged, as well.

The inventors of the present application use a simple thrust structure to replace the complicated wrapping-deploying mechanism used in U.S. Pat. No. 6,719,773 and eliminate the wrapping-deploying step of the operation method in U.S. Pat. No. 6,719,773. Meanwhile, unlike US patent publication 2004/0073308, the present invention does not need to crate a cavity in advance in the bone under treatment, and does not need to perform a deploying step required in the prior art. Furthermore, when necessary, the step of creating a channel in the bone under treatment can be omitted. Such a step of creating a channel is a necessary step in a similar operation according to the current technology. Thus, the medical implant according to the invention can simplify the operation procedures and achieve the advantages of shortening the operation time and providing faster recovery after operation.

### SUMMARY OF THE INVENTION

An objective of the present invention is to provide a medical implant equipped with a thrust part.

Another objective of the present invention is to provide a medical implant which can be implanted without the need of creating a cavity in advance in a bone under treatment.

Still an objective of the present invention is to provide a medical implant that a medical filling material can be inserted or infused through a tubular fitting.

Still a further objective of the present invention is to provide a vertebral implant equipped with a thrust part.

Still another objective of the present invention is to provide a vertebral implant that can be implanted without the need of creating a cavity in advance in the vertebral body under treatment.

Still a further objective of the present invention is to provide a vertebral body implant that a medical filling can be inserted or infused through a tubular fitting.

In order to accomplished the aforesaid objectives a medical implant constructed according to the present invention comprises:

a flexible container, a proximal end thereof being an open end, the flexible container having one layer of flexible surrounding wall or plural layers of flexible surrounding walls, each surrounding wall being provided with a plurality of through holes thereon through which an interior of the flexible container is in fluid communication with an exterior of the flexible container; a tubular fitting being connected to the open end of the flexible container for inserting or infusing a medical filling material into the flexible container through the tubular fitting; and a thrust part connected to the flexible surrounding wall(s) of the flexible container and at a proximal end of the flexible container.

The above-mentioned flexible container can be a flexible non-airtight filling member defined in the original invention of the present invention and related inventions disclosed in US patent publication Nos. 2004/0122455, and 2004/0210297. Such a flexible container can also be a flexible container described in the similar devices disclosed in, for examples the above-mentioned US patent/publications: US patent publication Nos. 2004/0073308, and 2006/0149379; and U.S. Pat. No. 6,719,773. The definitions and usages of the surrounding wall(s) and through holes of the flexible container can also be referred to the original invention of the present invention disclosed in US patent publication Nos. 2004/0122455, and 2004/0210297 and the related inventions, e.g. US patent publication Nos. 2004/0073308, and 2006/0149379; and U.S. Pat. No. 6,719,773.

The above-mentioned tubular fitting can be found in the original invention of the present invention and related inventions disclosed in US patent publication Nos. 2004/0122455, and 2004/0210297. Such a tubular fitting can also be a tubular fitting described in the similar devices disclosed in, for examples the above-mentioned US patent/publications: US patent publication Nos. 2004/0073308, and 2006/0149379; and U.S. Pat. No. 6,719,773.

The above-mentioned flexible container and tubular fitting can be assembled by a technique described in the original invention of the present invention or related inventions, or similar techniques described in, for examples the above-mentioned US patent/publications: US patent publication Nos. 2004/0073308, and 2006/0149379; and U.S. Pat. No. 6,719,773.

The above-mentioned medical filling material can be a medical material defined in the original invention of the present invention and related inventions disclosed in US patent publication Nos. 2004/0122455, and 2004/0210297; or a medical filling described in similar techniques, e.g. the above-mentioned US patent/publications: US patent publication Nos. 2004/0073308, and 2006/0149379; and U.S. Pat. No. 6,719,773. That is the medical filling material of the invention is a slurry prepared prior to operation, which then solidifies after implantation, or a bone graft in the form of solid granules.

The above-mentioned thrust part may comprise a fastener and a receiving member, in which one end of the receiving member can be inserted into the fastener and fastened therewith, in which the flexible surrounding wall(s) at the proximal end of the flexible container is fastened between the fastener and a receiving member. The structure of the thrust part and its method of combination with the flexible container are described in the following preferred embodiments.

The present invention further provides a medical kit comprising a medical implant and an infusion tool, said implant comprising:

a flexible container, a proximal end thereof being an open end, said flexible container having one layer of flexible surrounding wall or plural layers of flexible surrounding walls, each surrounding wall being provided with a plurality of through holes thereon through which an interior of the flexible container is in fluid communication with an exterior of the flexible container;

a tubular fitting being connected to the open end of the flexible container for inserting or infusing a medical filling material into the flexible container through the tubular fitting; and

a thrust part connected to the flexible surrounding wall(s) of the flexible container and at a proximal end of the flexible container, and

said infusion tool comprising:

an infusion tube; and

a syringe,

in which one end of the infusion tube is provided with external threads which are threadably connected to inner threads provided at a proximal end of the tubular fitting, and another end of the infusion tube is connected to said syringe, so that the infusion tube and the flexible container are in fluid communication with each other, and so that a medical filling material as a slurry in the syringe can be pushed and squeezed into the flexible container.

Preferably, the medical kit further comprises a thrust pin received in the flexible container and the tubular fitting, in which a distal end of the thrust pin contacts the thrust fitting to tension the flexible surrounding wall(s) of the flexible container.

### BRIEF DESCRIPTION OF THE DRAWINGS

FIG. 1a to FIG. 1e are schematic diagrams showing operation methods using a porous mesh device described in the prior art US patent/patent publications, wherein FIGS. 1a and 1b do not use a pre-formed cavity and FIGS. 1c to 1e use a pre-formed cavity in the vertebral body.

FIG. 2 is a schematic perspective diagram showing a flexible non-airtight medical filling device (a medical kit) according to a preferred embodiment of the present invention.

FIG. 3a is a schematic perspective diagram showing a medical implant before being assembled according to a preferred embodiment of the present invention.

FIG. 3b is a schematic perspective diagram showing the medical implant in FIG. 3a after being assembled.

FIG. 3c is a schematic partial cross-sectional diagram of a medical implant according to a preferred embodiment of the present invention, which shows the thrust part 31 is connected to the distal end of the flexible container 20.

FIG. 3d is a schematic partial cross-sectional diagram of a medical implant according to another preferred embodiment of the present invention, which shows the thrust part 31 is connected to the distal end of the flexible container 20.

FIG. 4a is a schematic perspective diagram showing the thrust part 31 in FIG. 3d before being assembled.

FIG. 4b is a schematic perspective diagram showing the thrust part 31 in FIG. 3d after being assembled.

FIG. 5a to FIG. 5d are schematic diagrams showing operation procedures for implanting the medical implant of the present invention into a vertebral body.

### DETAILED DESCRIPTION OF THE INVENTION

Preferred embodiments of the present invention in conjunction with the accompanying drawings will be described in the following to further elaborate the present invention:

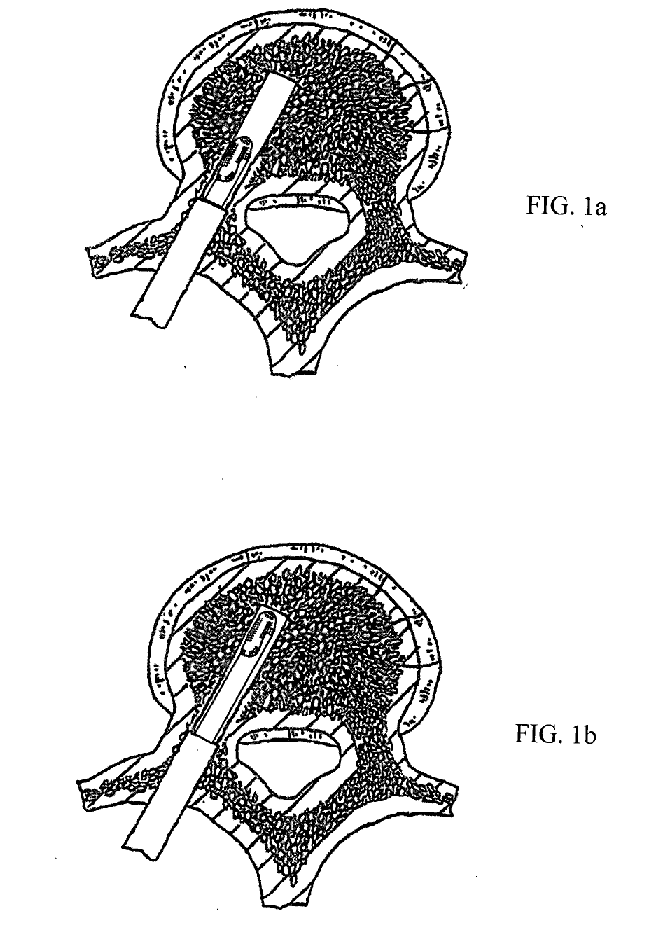

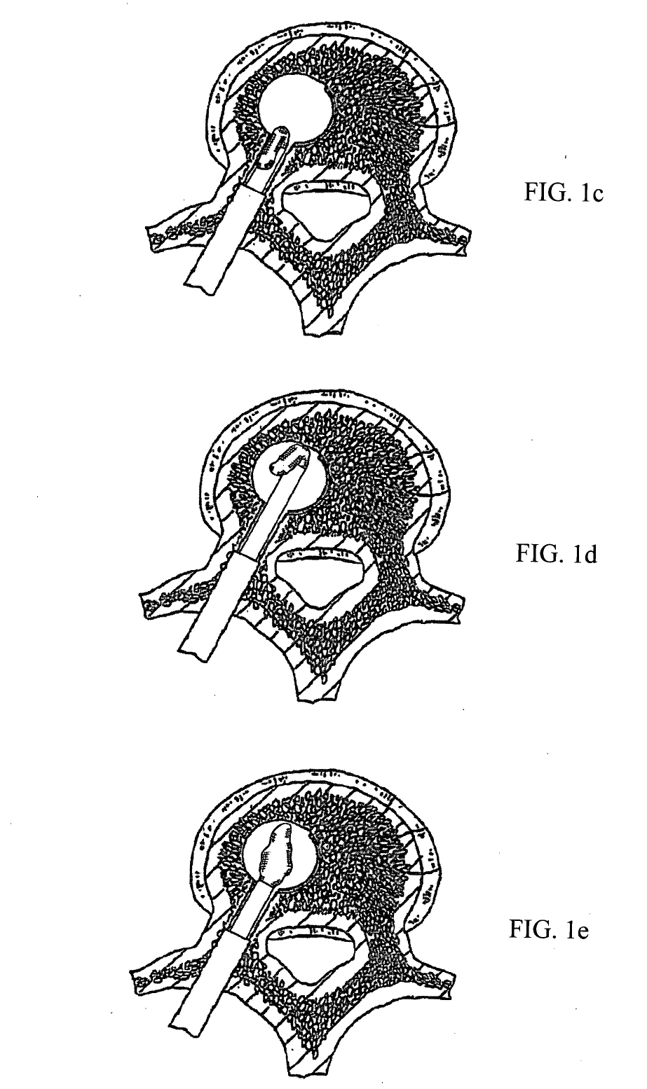

FIG. 1a to FIG. 1b show that a cavity needed to be created in the vertebral body in advance for the porous mesh device disclosed in US patent publication Nos. 2004/0073308 and 2006/0149379 to be successfully deployed in the vertebral body. FIG. 1c to FIG. 1e show that after a cavity was created, the porous mesh device is pushed to a pre-determined position (FIG. 1c), and then the pushing is continued until the porous mesh device is deployed (FIG. 1d). Next, the porous mesh device is returned to the pre-determined position (FIG. 1e), so that the porous mesh device in the cavity of the vertebral body deploys voluntarily into a shape for facilitating the subsequent filling operation. Accordingly, a complicated wrapping-deploying mechanism described in U.S. Pat. No. 6,719,773 could be avoided.

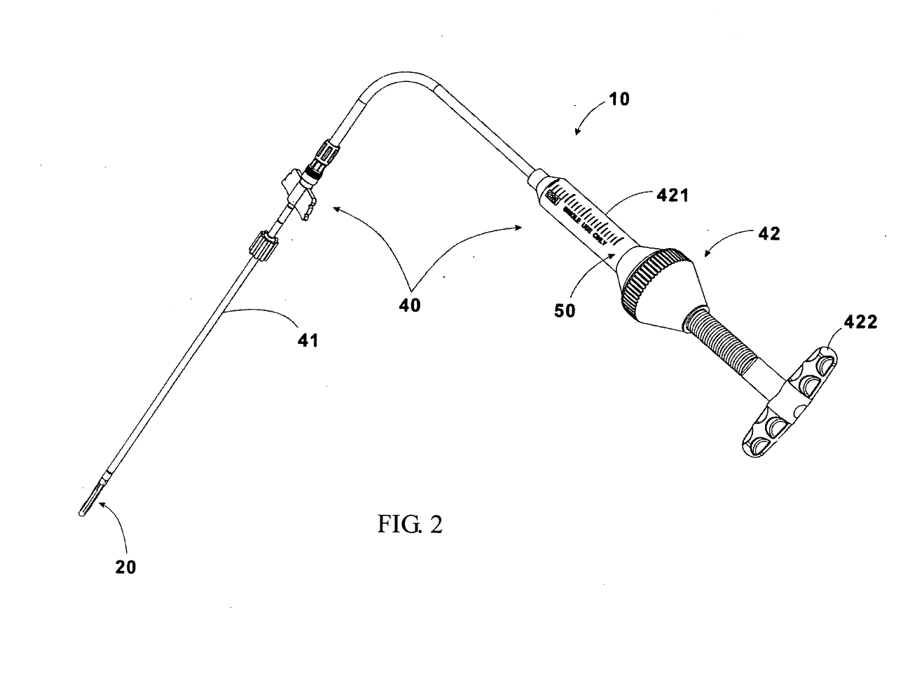

FIG. 2 shows a flexible non-airtight medical filling device 10, i.e. the above-mentioned medical kit of the present invention, which includes: a flexible non-airtight filling member 20 (i.e. the flexible container) and a matching infusion tool 40. The infusion tool 40 includes an infusion tube 41, a syringe 42, and a medical filling material 50 which is prepared into a slurry form and loaded in the syringe 42.

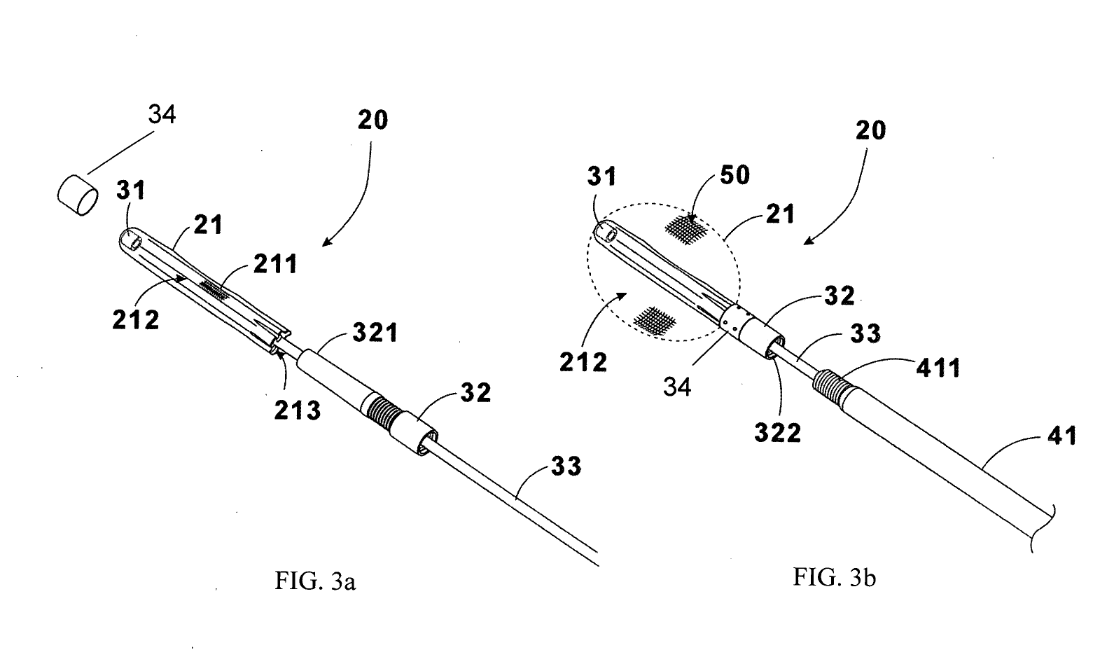

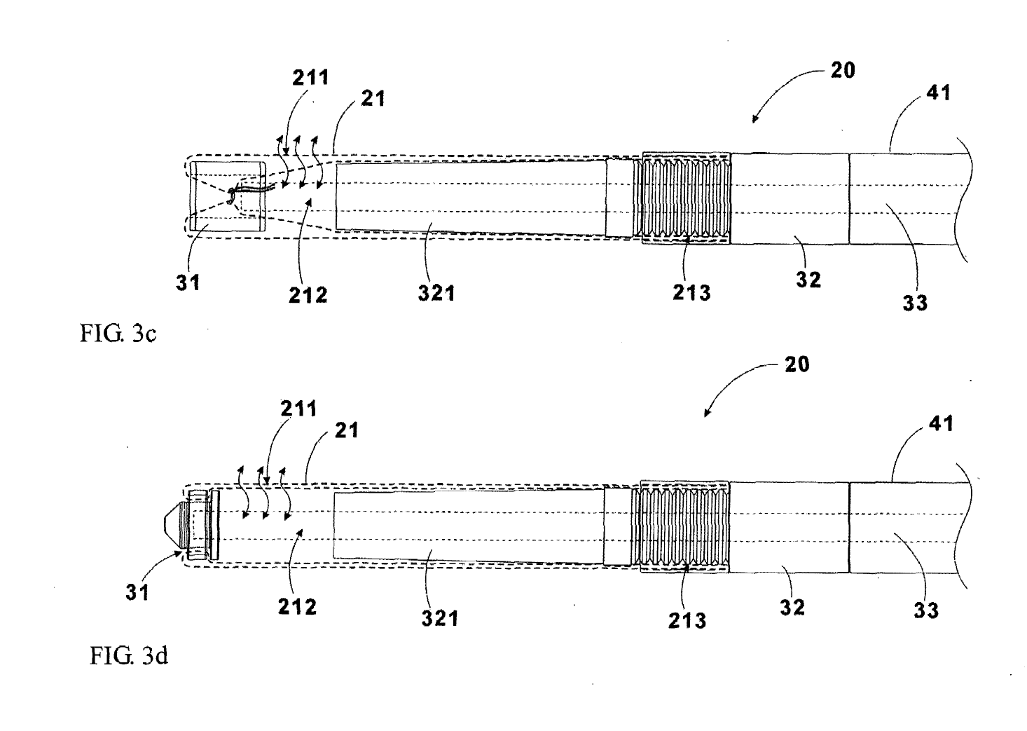

FIG. 3a to FIG. 3d show that the flexible non-airtight filling member 20 included one or plural overlapping layers of flexible non-airtight surrounding wall(s) 21. The surrounding wall(s) 21 is provided with through holes 211 with a mesh number greater than 100, which can be a single-layered or multi-layered, stacked deformable surrounding wall(s).

The surrounding wall(s) 21 is formed integrally or with plural adjacent walls with adjacent peripheries thereof joined with a housing part 212 and an infusion part 213 (open end). The surrounding wall(s) 21 of the flexible non-airtight filling member 20 can be folded and compressed in advance, and the slurry medical filling material 50 is inserted or infused into the housing part 212 through the infusion part 213, so that the folded and compressed surrounding wall(s) 21 is restored and inflated. The through holes 211 enable the internal surface and the external surface of the housing part 212 surrounded by the surrounding wall(s) to communicate with each other.

Moreover, the filling member 20 further includes a thrust part 31 and a nozzle 32 (i.e. a tubular fitting). Furthermore, in order to match the thrust part 31, a thrust pin 33 is used to movably push the thrust part 31.

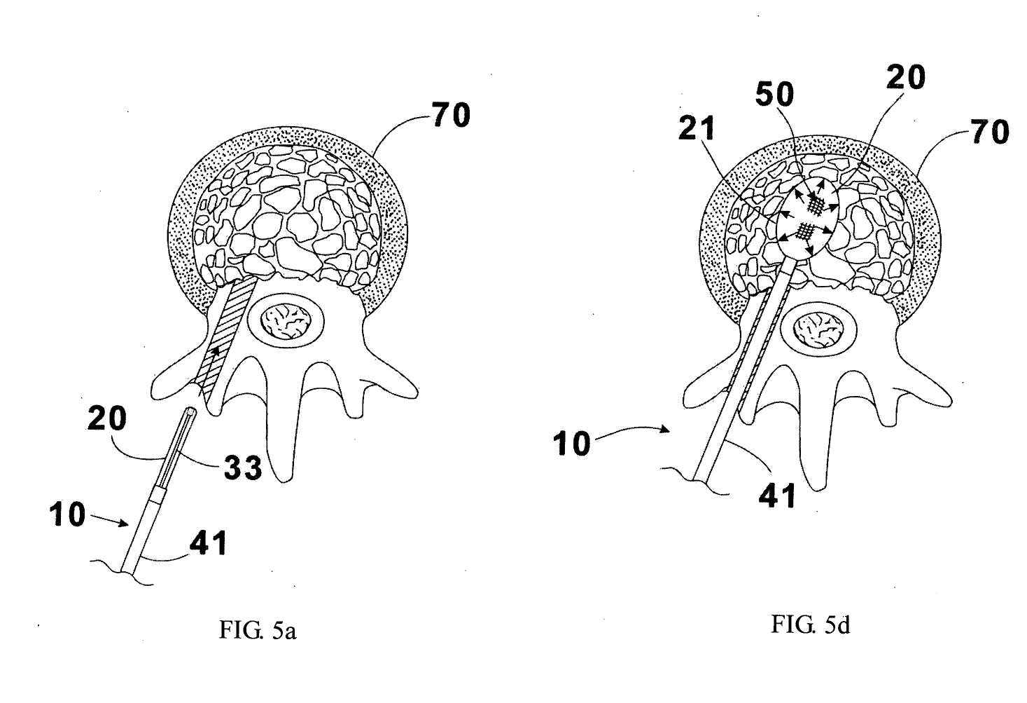

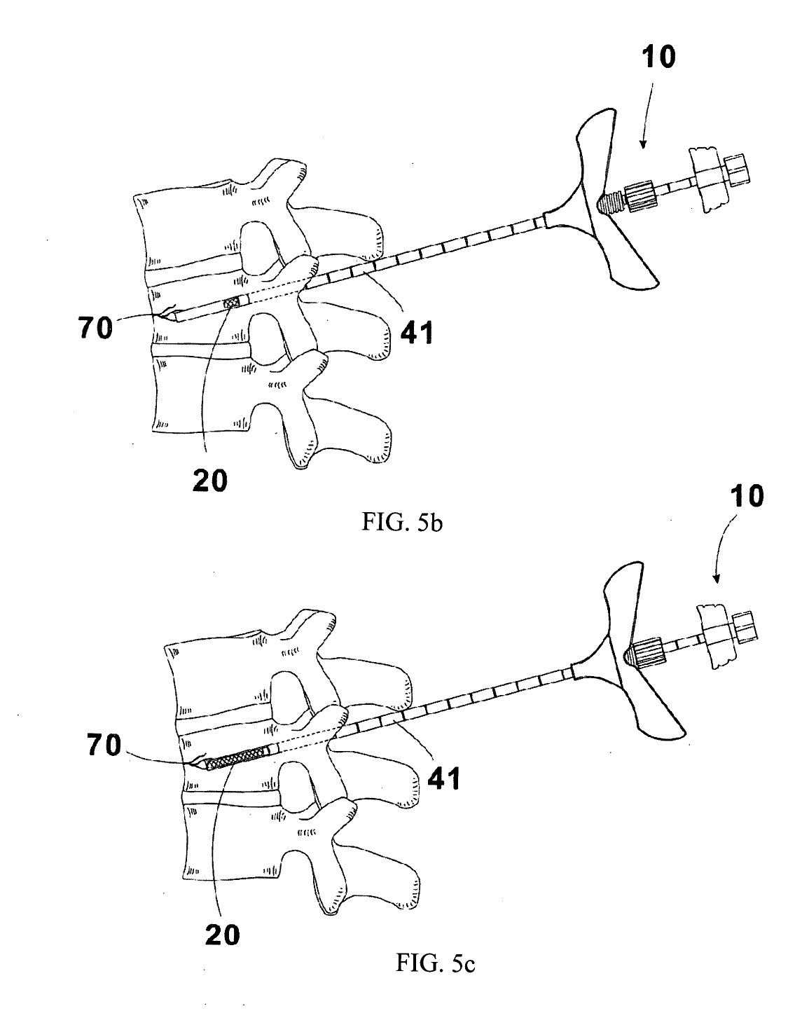

An infusion part 213 of the filling member 20 is fixedly connected to the nozzle 32 with a retainer ring 34. One end 321 of the nozzle 32 is housed in the housing part 212. The surrounding wall(s) 21 opposite to to the infusion part 213 is connected with the thrust part 31, and the thrust pin 33 is inserted into the nozzle 32 and the infusion part 213 and pressed forwardly onto the thrust part 31, so that the surrounding wall(s) 21 of the filling member 20 is tensioned into a linear shape, and thus the filling member 20 can resist a counter force when a force is applied to insert the filling member 20 into a vertebral body (70 in FIG. 5a-5d) without the need of pre-twisting the filling member 20 into a force-receiving shape in advance.

Referring to FIG. 2 and FIG. 3b, one end of the infusion tube 41 is provided with a plurality of external threads 411 corresponding to a plurality of inner threads 322 on the inner surface of the nozzle 32, and they are threadably connected, communicating the infusion tube 41 with the housing part 212 of the flexible non-airtight filling member 20. Another end of the infusion tube 41 is connected to the syringe 42. The syringe 42 includes a housing cylinder 421 and a push rod 422. The housing cylinder 421 is able to store the slurry medical filling material 50, and the push rod 422 is used to squeeze the slurry medical filling material 50 into the housing part 212 of the filling member 20 through the infusion tube 41 by a reciprocation motion.

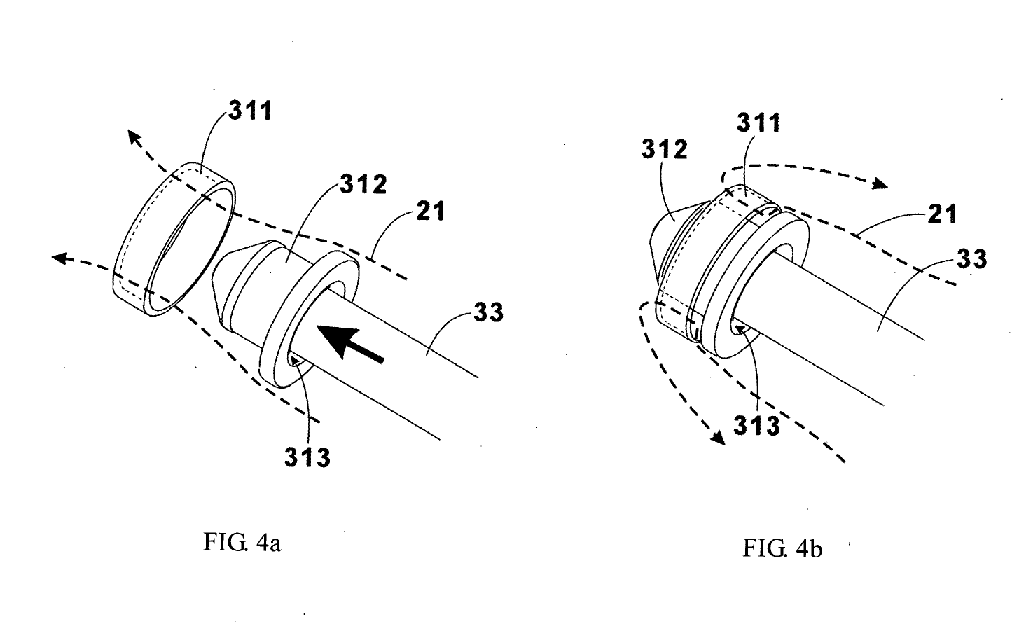

Referring to FIG. 4a and FIG. 4b, the thrust part 31 has a fastener 311 and a receiving member 312. One end of the receiving member 312 is able to be inserted into the fastener 311 and engaged therewith. The other end of the receiving member 312 has a recess 313. The thrust pin 33 is able to be inserted into the recess 313, and is used to push the receiving member 312 forward.

Referring to FIG. 5a to FIG. 5d, the operator can directly insert the “flexible” non-airtight filling member 20 of the flexible non-airtight medical filling device 10 into the vertebral body 70 by using the infusion tube 41 and the thrust pin 33 received therein, which has been formed with a drilling hole, without the need of creating a cavity in the vertebral body in advance and without twisting the expandable structure 56 disclosed in U.S. Pat. No. 6,719,773 into a tight force-receiving shape before being inserted.

The filling member 20 is able to be squeezed by the slurry medical filling material 50 and expanded in the vertebral body 70. Furthermore, under the restriction of the flexible non-airtight surrounding wall(s) 21, the slurry medical filling material 50 is able to be stably and gradually filled in the filling member 20.

1. A medical implant comprising:
a flexible container, a proximal end thereof being an open end, said flexible container having one layer of flexible surrounding wall or plural layers of flexible surrounding walls, each surrounding wall being provided with a plurality of through holes thereon through which an interior of the flexible container is in fluid communication with an exterior of the flexible container;
a tubular fitting being connected to the open end of the flexible container for inserting or infusing a medical filling material into the flexible container through the tubular fitting; and
a thrust part connected to the flexible surrounding wall(s) of the flexible container and at a proximal end of the flexible container.

2. The implant as claimed in claim 1, in which the thrust part comprises a fastener and a receiving member, in which the fastener fastens the receiving member to the flexible surrounding wall(s) at the proximal end of the flexible container.

3. The implant as claimed in claim 1, in which the through holes have a size smaller that of a mesh number of 100.

4. The implant as claimed in claim 3, in which the thrust part comprises a fastener and a receiving member, in which the fastener fastens the receiving member to the flexible surrounding wall(s) at the proximal end of the flexible container.

5. The implant as claimed in claim 3, in which the tubular fitting is provided with threads at a proximal end thereof for the convenience of mounting/dismounting of a tool for infusing the medical filling material.

6. The implant as claimed in claim 3, in which the medical filling material is in the form of slurry prepared prior to being infused into the flexible container through the tubular fitting, which solidifies after being infused.

7. The implant as claimed in claim 6, i in which the thrust part comprises a fastener and a receiving member, in which the fastener fastens the receiving member to the flexible surrounding wall(s) at the proximal end of the flexible container.

8. The implant as claimed in claim 6, in which the tubular fitting is provided with threads at a proximal end thereof for the convenience of mounting/dismounting of a tool for infusing the medical filling material.

9. A medical kit comprising a medical implant and an infusion tool, said implant comprising:
a flexible container, a proximal end thereof being an open end, said flexible container having one layer of flexible surrounding wall or plural layers of flexible surrounding walls, each surrounding wall being provided with a plurality of through holes thereon through which an interior of the flexible container is in fluid communication with an exterior of the flexible container;
a tubular fitting being connected to the open end of the flexible container for inserting or infusing a medical filling material into the flexible container through the tubular fitting; and
a thrust part connected to the flexible surrounding wall(s) of the flexible container and at a proximal end of the flexible container, and
said infusion tool comprising:
an infusion tube; and
a syringe,
in which one end of the infusion tube is provided with external threads which are threadably connected to inner threads provided at a proximal end of the tubular fitting, and another end of the infusion tube is connected to said syringe, so that the infusion tube and the flexible container are in fluid communication with each other, and so that a medical filling material as a slurry in the syringe can be pushed and squeezed into the flexible container.

10. The medical kit further comprising a thrust pin received in the flexible container and the tubular fitting, in which a distal end of the thrust pin contacts the thrust fitting to tension the flexible surrounding wall(s) of the flexible container.
